(19) United States
(12) Patent Application Publication
(10) Pub. No.: US 2026/0124441 A1
(43) Pub. Date: May 7, 2026

(54) CARDIOVASCULAR SUPPORT PUMP HAVING AN IMPELLER WITH A VARIABLE FLOW AREA

(71) Applicant: Kardion GmbH, Stuttgart (DE)

(72) Inventors: Johannes Bette, Balingen (DE); Vladimir Popov, Stuttgart (DE); Marvin Mitze, Stuttgart (DE)

(21) Appl. No.: 19/440,043

(22) Filed: Jan. 5, 2026

Related U.S. Application Data
(63) Continuation of application No. 18/026,082, now Pat. No. 12,515,036.
(60) Provisional application No. 63/078,207, filed on Sep. 14, 2020.

(51) Int. Cl. A61M 60/221; A61M 60/13; A61M 60/416; A61M 60/422; A61M 60/806; A61M 60/857

(52) U.S. Cl. CPC A61M 60/221; A61M 60/13; A61M 60/416; A61M 60/422; A61M 60/806; A61M 60/857

(57) ABSTRACT

The present disclosure is directed generally to mechanical cardiovascular support systems used in the medical field to assist the movement of blood. In particular the present disclosure is directed to an impeller having features that allow improved performance. An annular flow area around a rotating impeller may be variable along the axial length of the impeller. A first radial gap, between a distal region of the impeller and a surrounding tubular housing, may be greater or smaller than a second radial gap, between a proximal region of the impeller and the surrounding tubular housing.

### CROSS-REFERENCE TO RELATED APPLICATIONS

This application is a continuation application of U.S. Non-Provisional application Ser. No. 18/026,082, filed Mar. 13, 2023, which is claims the priority benefit of PCT No. PCT/US2021/071419, filed Sep. 10, 2021, which claims the priority benefit of U.S. Provisional Patent Application No. 63/078,207, filed Sep. 14, 2020, the entire disclosure of which are hereby incorporated by reference herein in their entireties.

### BACKGROUND

### Technical Field

The present disclosure is directed generally to mechanical cardiovascular support systems used in the medical field to assist the movement of blood. In particular, the present disclosure is directed to mechanical cardiovascular support devices having features that allow improved performance such as optimized flow area between impeller blades or shafts or other components and a surrounding inlet tube, or optimized space between certain stationary and moving parts.

### Description of the Related Art

Mechanical circulatory support devices are used to assist a patient's heart with moving blood through the body. They often consist of an electrical motor, a tubular inflow canula with an inflow area at the distal end and an outflow area at the proximal end, and an impeller rotated by the motor that moves blood through the tubular inflow cannula and out through the outflow area. A particular design challenge is to optimize blood flow while minimizing risk of damage to the device or to the blood. A higher blood flow than what is provided by current devices may improve outcomes for mild cardiogenic shock patients or high-risk percutaneous coronary intervention patients. However, increasing flow may also increase risk of hemolysis or device damage.

Thus, there is a need for improved mechanical circulatory support designs that can allow for greater blood flow while avoiding risk of hemolysis or device damage, among other improvements.

### SUMMARY

This disclosure is related to mechanical circulatory support systems having an impeller with a variable outer diameter or having a variable radial gap between the impeller blades or other components of and impeller and a tubular cannula containing the impeller. The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices, and methods for mechanical circulatory support systems.

The following disclosure describes non-limiting examples of some embodiments of mechanical circulatory support devices. For instance, other embodiments of the disclosed systems and methods may or may not include the features described herein. Moreover, disclosed advantages and benefits can apply only to certain embodiments and should not be used to limit the disclosure.

One aspect of the disclosure is a mechanical circulatory support device including: a tubular cannula including an inflow region, an outflow region, and a distal impeller region, and an impeller having a proximal end and a distal end, the impeller being positioned within the tubular cannula at least partly in the outflow region and at least partly in the distal impeller region, wherein a radial gap between the impeller and an inner surface of the tubular cannula varies in size between the proximal end and the distal end, wherein the size of the radial gap at a particular location between the proximal end and the distal end includes a radial distance between a maximum impeller width and the inner surface of the tubular cannula at the particular location.

A 2^{nd} aspect of the disclosure relates to the device of aspect 1, wherein the impeller includes a distally tapered section tapering distally from a first diameter to a distal diameter, wherein the distal diameter is less than the first diameter.

A 3^{rd} aspect of the disclosure relates to the device of aspect 2, wherein the impeller includes a constant diameter section extending proximally from the distally tapered section, the constant diameter section having the first diameter.

A 4^{th} aspect of the disclosure relates to the device of aspect 3, wherein the constant diameter section extends at least partially within the distal impeller region and at least partially into the outflow region.

A 5^{th} aspect of the disclosure relates to the device of either aspect 3 or aspect 4, wherein the constant diameter section extends from the distally tapered section to the proximal end of the impeller.

A 6^{th} aspect of the disclosure relates to the device of either aspect 3 or aspect 4, wherein the impeller includes a proximal narrowed diameter section proximal to the constant diameter section, the proximal narrowed diameter section having a narrowed diameter less than the first diameter.

A 7^{th} aspect of the disclosure relates to the device of aspect 6, wherein the narrowed diameter is smaller than the first diameter by between 0.15 mm and 0.35 mm.

An 8^{th} aspect of the disclosure relates to the device of any one of aspects 6 to 7, wherein the radial gap at the proximal narrowed diameter section is about 0.20 mm greater than a minimum radial gap in the distal impeller region.

A 9^{th} aspect of the disclosure relates to the device of any one of aspects 6 to 7, wherein the radial gap at the proximal narrowed diameter section is about 0.125 mm greater than a minimum radial gap in the distal impeller region.

A 10^{th} aspect of the disclosure relates to the device of any one of aspects 2 to 9, wherein the radial gap is between 0.095 mm and 0.125 mm at a location of the distal diameter.

An 11^{th} aspect of the disclosure relates to the device of any one of aspects 2 to 10, wherein the radial gap is between 0.060 mm and 0.090 mm at the location of the first diameter.

A 12^{th} aspect of the disclosure relates to the device of any one of aspects 2 to 11, wherein the distal diameter is between 0.40 mm and 0.100 mm less than the first diameter.

A 13^{th} aspect of the disclosure relates to the device of any one of aspects 2 to 12, wherein a slope of the tapered section is about 0.0117.

A 14^{th} aspect of the disclosure relates to the device of any one of aspects 2 to 13, wherein an inner diameter of the tubular cannula is between 4.39 mm and 4.45 mm.

A 15^{th} aspect of the disclosure relates to the device of any one of aspects 2 to 14, wherein a length of the impeller is between 7.5 mm to 8.5 mm.

A 16^{th} aspect of the disclosure relates to the device of any one of aspects 2 to 15, wherein the proximal end of the impeller is mounted to a drive shaft.

A 17^{th} aspect of the disclosure relates to the device of any one of aspects 2 to 16, wherein the distal end of the impeller is a free end.

An 18^{th} aspect of the disclosure relates to the device of any one of aspects 2 to 17, further including a motor housing, wherein the motor housing is coupled to a proximal end of the inlet cannula.

A 19^{th} aspect of the disclosure relates to the device of aspect 1, further including a driven magnetic rotor, wherein the impeller is coupled to the driven magnetic rotor.

A 20^{th} aspect of the disclosure relates to the device of aspect 19, further including a bearing configured to hold the distal end of the impeller in an axial center of the tubular cannula.

A 21^{st} aspect of the disclosure relates to the device of aspect 20, wherein the bearing includes a plurality of spokes connecting the bearing to the tubular cannula and configured to maintain the position of the bearing with respect to the tubular cannula.

A 22^{nd} aspect of the disclosure relates to the device of any one of aspects 19 to 21, wherein the driven magnetic rotor is positioned at least partially in the outflow region.

A 23^{rd} aspect of the disclosure relates to the device of any one of aspects 19 to 22, wherein the radial gap between the impeller and the inner surface of the tubular cannula is greater in the outflow region than in the distal impeller region.

A 24^{th} aspect of the disclosure relates to the device of any one of aspects 19 to 23, wherein the radial gap between the impeller and the inner surface of the tubular cannula is between 2 and 3 times greater in at least a portion of the outflow region than at the distal impeller region.

A 25^{th} aspect of the disclosure relates to the device of any one of aspects 19 to 22, wherein the impeller includes a constant diameter.

A 26^{th} aspect of the disclosure relates to the device of any one of aspects 19 to 25, wherein the impeller includes a first diameter within the distal impeller region and the driven magnetic rotor includes second diameter within the outflow region smaller than the first diameter.

A 27^{th} aspect of the disclosure relates to the device of aspect 26, wherein the radial gap between the impeller and the inner surface of the tubular cannula at a location of the first diameter is larger than a second radial gap between the driven magnetic rotor and the tubular cannula at a location of the second diameter.

A 28^{th} aspect of the disclosure relates to the device of aspect 27, wherein the radial gap between the impeller and the inner surface of the tubular cannula at the location of the first diameter is between 0.065 mm to 0.150 mm.

A 29^{th} aspect of the disclosure relates to the device of either aspect 27 or 28, wherein the second radial gap is between 2 and 3 times larger than the radial gap between the impeller and the inner surface of the tubular cannula at the location of the first diameter.

A 30^{th} aspect of the disclosure is a mechanical circulatory support device including: a tubular cannula including an inflow region, an outflow region, and a distal impeller region, and an impeller positioned in the tubular cannula partly in the outflow region and partly in the distal impeller region, wherein a first radial gap between the impeller and the distal impeller region is smaller than a second radial gap between the impeller and the outflow region of the tubular cannula.

A 31^{st} aspect of the disclosure relates to the device of aspect 30, wherein the outflow region includes one or more struts.

A 32^{nd} aspect of the disclosure relates to the device of any one of aspects 30 to 31, wherein the impeller has a first diameter in the distal impeller region and a second diameter in the outflow region, wherein the second diameter is smaller than the first diameter.

A 33^{rd} aspect of the disclosure relates to the device of aspect 32, wherein the first diameter is in a range of 3.8 mm to 5.92 mm.

A 34^{th} aspect of the disclosure relates to the device of aspect 32 or aspect 33, wherein the second diameter is in a range of 0.5 mm to 5.92 mm.

A 35^{th} aspect of the disclosure relates to the device of any one of aspects 32 to 34, wherein a transition from the first diameter to the second diameter is a step transition.

A 36^{th} aspect of the disclosure relates to the device of any one of aspects 32 to 34, wherein a transition from the first diameter to the second diameter is a sloped transition.

A 37^{th} aspect of the disclosure relates to the device of any one of aspects 32 to 34, wherein a transition from the first diameter to the second diameter is a curved transition.

A 38^{th} aspect of the disclosure relates to the device of any one of aspects 30 to 31, wherein the impeller has a constant diameter.

A 39^{th} aspect of the disclosure relates to the device of any one of aspects 30 to 31 or 38, further including an insert sleeve positioned in the distal impeller region.

A 40^{th} aspect of the disclosure relates to the device of aspect 39, wherein the insert sleeve includes a chamfered distal end and a chamfered proximal end.

A 41^{st} aspect of the disclosure relates to the device of any one of aspects 39 or 40, wherein the insert sleeve is made from PEEK.

A 42^{nd} aspect of the disclosure relates to the device of any one of aspects 39 to 41, wherein the insert sleeve includes an inner surface with a lubricious coating.

A 43^{rd} aspect of the disclosure relates to the device of any one of aspects 30 to 42, wherein the first radial gap is in a range of 0.04 mm to 0.5 mm.

A 44^{th} aspect of the disclosure relates to the device of any one of aspects 30 to 43, wherein the second radial gap is in a range of 0.04 mm to 3 mm.

A 45^{th} aspect of the disclosure relates to the device of any one of aspects 30 to 44, wherein the impeller is connected to a drive shaft of a motor.

A 46^{th} aspect of the disclosure relates to the device of any one of aspects 30 to 44, wherein the impeller is magnetically coupled to a motor.

A 47^{th} aspect of the disclosure relates to the device of any one of aspects 30 to 46, wherein the impeller includes at least two impeller blades.

A 48^{th} aspect of the disclosure relates to the device of any one of aspects 30 to 47 in combination with aspect 31, wherein the struts have a thickness that is greater than a thickness of the tubular cannula.

A 49^{th} aspect of the disclosure relates to the device of any one of aspects 30 to 37, wherein the impeller includes impeller blades in the distal impeller region, and wherein the device further includes a radially symmetric conical form in the outflow region.

A 50^{th} aspect of the disclosure relates to the device of aspect 49, wherein the radially symmetric conical form has an electropolished surface.

A 51^{st} aspect of the disclosure relates to the device of any one of aspects 49 or 50, wherein the radially symmetric conical form includes a concave surface.

A 52^{nd} aspect of the disclosure relates to the device of any one of aspects 49 to 51, wherein the radially symmetric conical form includes a convex surface.

A 53^{rd} aspect of the disclosure relates to the device of aspect 52 in combination with aspect 51, wherein the convex surface is proximal to the concave surface.

A 54^{th} aspect of the disclosure relates to the device of any one of aspects 49 to 53, wherein the radially symmetric conical form is affixed to or part of an impeller shaft.

A 55^{th} aspect of the disclosure relates to the device of any one of aspects 49 to 53, wherein the radially symmetric conical form is affixed to or part of a motor housing or the tubular cannula.

A 56^{th} aspect of the disclosure relates to the device of aspect 55, wherein an impeller shaft of the impeller passes through a lumen in the conical form.

A 57^{th} aspect of the disclosure relates to the device of aspect 56, wherein a bearing is positioned between the impeller shaft and the conical form.

A 58^{th} aspect of the disclosure relates to the device of aspect 56 or 57, wherein a seal is positioned between the impeller shaft and the conical form.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a schematic illustration of a mechanical circulatory support (MCS) device.

FIG. 2A is a schematic illustration of a cutaway view of a portion of an MCS device.

FIG. 2B is a cross section of the distal impeller region of FIG. 2A.

FIG. 2C is a cross section of the outflow region of FIG. 2A.

FIG. 3 is a schematic illustration of an MCS device having a consistent radial gap in the distal impeller region and outflow region.

FIG. 4 is a schematic illustration of an MCS device having a minimalized radial gap in the distal impeller region and a larger radial gap in the outflow region.

FIG. 5 is a schematic illustration of an MCS device having a minimalized radial gap in the distal impeller region and a larger radial gap in the outflow region with a sloped transition.

FIG. 6 is a schematic illustration of an MCS device having an impeller with a consistent diameter and an insert sleeve to minimize the radial gap in the distal impeller region.

FIG. 7 is a schematic illustration of an MCS device having an impeller with impeller blades in a distal impeller region and no impeller blades in the outflow region.

FIG. 8 is a schematic illustration of an MCS device having an impeller with impeller blades in a distal impeller region and a conical form in the outflow region connected to the impeller.

FIG. 9 is a schematic illustration of an MCS device having an impeller with impeller blades in a distal impeller region and a conical form in the outflow region that remains stationary with respect to the motor housing and tubular cannula.

FIG. 10 is a schematic illustration of an MCS device having an impeller having a tapered distal section.

FIG. 11 is a schematic illustration of an MCS device having an impeller having a tapered distal section and a proximally narrowed section.

FIG. 12A is a schematic illustration of an MCS device having a variable radial gap between an impeller and a surrounding cannula or impeller housing and having a driven magnetic rotor.

FIG. 12B is a close up view of a portion of FIG. 12A.

### DETAILED DESCRIPTION

The disclosure herein is related to mechanical circulatory support devices having features that may optimize the ability to move blood while minimizing risk of damage to the device or to the blood. In particular, the disclosure relates to features of an impeller or components intended to rotate and their relationship with a tubular inflow cannula or components intended to remain stationary and their outflow region. The following detailed description is directed to certain specific embodiments. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings.

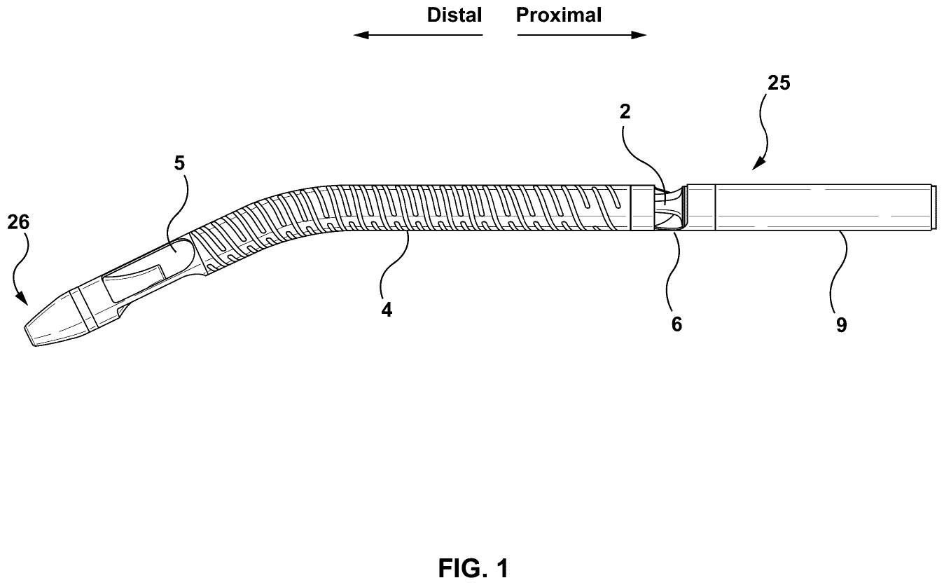

As shown in FIG. 1, a mechanical circulatory support (MCS) device may include an inlet tube or tubular inflow cannula 4 having a proximal end 25 and a distal end 26, an inflow region 5 at the distal end, an outflow region 6 at the proximal end, and a motor 9 for driving an impeller 2 that is positioned at least partially within the tubular canula. The rotating impeller draws blood into the stationary inflow canula 4 through the inflow region 5 and pushes the blood out of the device at least partially through the outflow region 6.

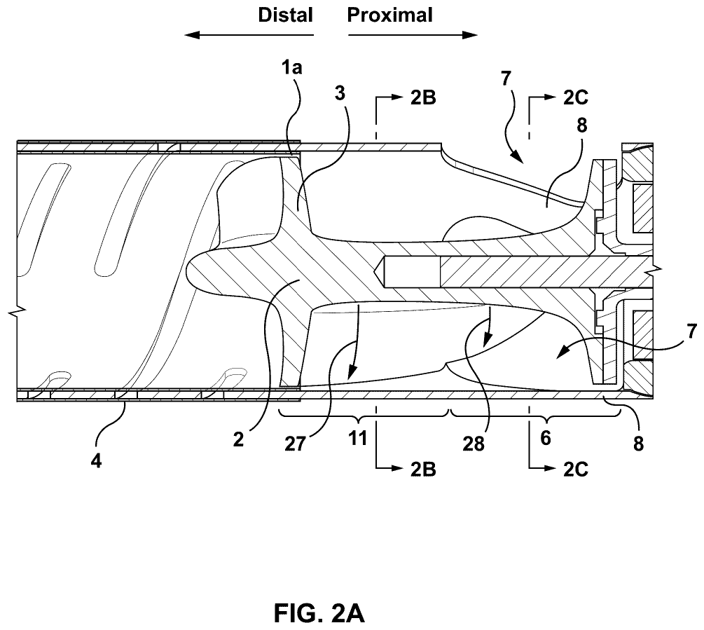

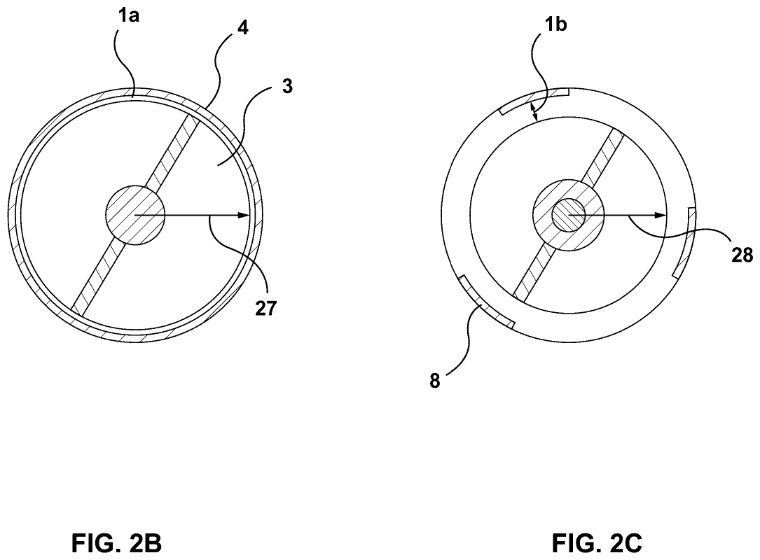

FIG. 2A is a close-up cut away view of a section of the MCS device of FIG. 1. The outflow region 6 may include struts 8 that connect the tubular inlet canula 4 to the motor housing 9. For example, an MCS device may have at least two struts 8 (e.g., three, four, five struts). The outflow region 6 has openings or windows 7 in the tubular canula defined by the struts 8. The inside or outside edges of the openings or the cut face of the openings 7 may be rounded (e.g., having a diameter of curvature equal to the thickness of the tubular inflow cannula, which may be in a range of 0.10 to 0.15 mm). There is a very small radial gap 1a between the outer edges of the impeller blades 3 and the inner surface of the tubular inflow cannula 4. The rotating edges of the impeller blades 3 should not contact the tubular canula 4 so as to avoid mechanical wear, friction, loss of impeller speed, increase in motor draw, generation of heat or other unwanted effects. However, it can be advantageous to have a minimal radial gap 1a between the impeller blades 3 and the inner surface of the cannula 4 in order to maximize efficiency and reduce hemolysis. Hemolysis is a form of blood damage that can be caused by hydraulic shear stress applied to the blood cells. Hemolysis may be characterized by damaged hemoglobin and a measure of hemolysis may include hemolysis damage potential (HDP), which corresponds to the ratio of the free hemoglobin to the total amount of hemoglobin in a blood sample. The amount of hemolysis may be dependent on several factors, one of which can be impeller speed. Thus, generally speaking, the higher the efficiency, or flow rate generated by a given impeller speed, the higher the flow rate may be while minimizing hemolysis. An effect of minimizing the radial gap 1a may include minimizing reflow, a factor influencing efficiency. Consequently, the smaller the radial gap 1a between the impeller blades and the inner surface of the tubular cannula 4 is, a higher flow rate to hemolysis damage potential may be realized facilitating the production of a sufficient flow rate while causing acceptably low hemolysis. This applies to a distal impeller region 11, a region distal to the outflow region 6 where the impeller blades rotate within a stiff, section of the tubular cannula 4, for example wherein laser cuts are not made to increase stiffness, wherein apart from the minimal radial gap the blood must flow in a space defined by the inner surface of the tubular cannula and the impeller. With a sufficient speed the helical pitch of the rotating impeller blades in the confined space defined by the tubular cannula generates a hydraulic operating point that causes the blood to flow through the distal impeller region 11 toward the outflow region 6. The displacement of blood creates a pressure differential that pulls blood into the inflow region 5 of the tubular cannula 4.

In the outflow region 6, the rotating impeller blades direct blood flow out of the outflow windows 7. In the outflow region 6 where the tubular cannula 4 has outflow openings or windows 7 defined by struts 8, minimizing a radial gap 1b between the impeller blades 3 and the inner surface of struts 8 can contribute less to efficiency compared to the distal impeller region 11 because of flow disturbances which occur as an interaction between blood flow and the struts 8. In some embodiments, minimizing the radial gap 1b in the outflow region 6 may undesirably increase the hemolysis damage potential, in part due to hydraulic shear forces applied to the blood cells in the space 1b between the impeller blades and the struts. Furthermore, minimizing the radial gap 1b may pose challenges of manufacturing very small tolerances that can increase risk of the blades and struts contacting during use, which may lead to material wear, increased motor current, heat production, or other device damage as well as increased hemolysis. Thus, an aspect of this disclosure relates to designs of MCS devices wherein a first radial gap 1a between impeller blades and the inner surface of the tubular cannula in the distal impeller region 11 is minimized (e.g., in a range of 0.04 mm to 0.5 mm), and a second radial gap 1b between impeller blades and the inner surface of the struts 8 is larger (e.g., in a range of 0.04 mm to 3 mm and larger than the first radial gap) than the first radial gap, which may optimize efficiency and reduce the risk of damaging blood or the device itself during use.

One way to increase the second radial gap 1b is to decrease the diameter of the impeller blades in the outflow region 6 compared to the diameter of the impeller blades in the distal impeller region 11, while maintaining a consistent tubular cannula inner diameter. With this approach the larger the radial gap 1b is, the smaller the diameter of the impeller blades in the outflow region is. FIG. 2A shows an example of an impeller 3 having a first radius 27 in the distal impeller region 11 and a second radius 28 in the outflow region 6. FIG. 2B is a cross sectional view of FIG. 2A at the distal impeller region 11 and FIG. 2C is a cross sectional view of FIG. 2A at the outflow region 6. Impeller radius 27 is larger than impeller radius 28 and consequently radial gap 1a is smaller than radial gap 1b.

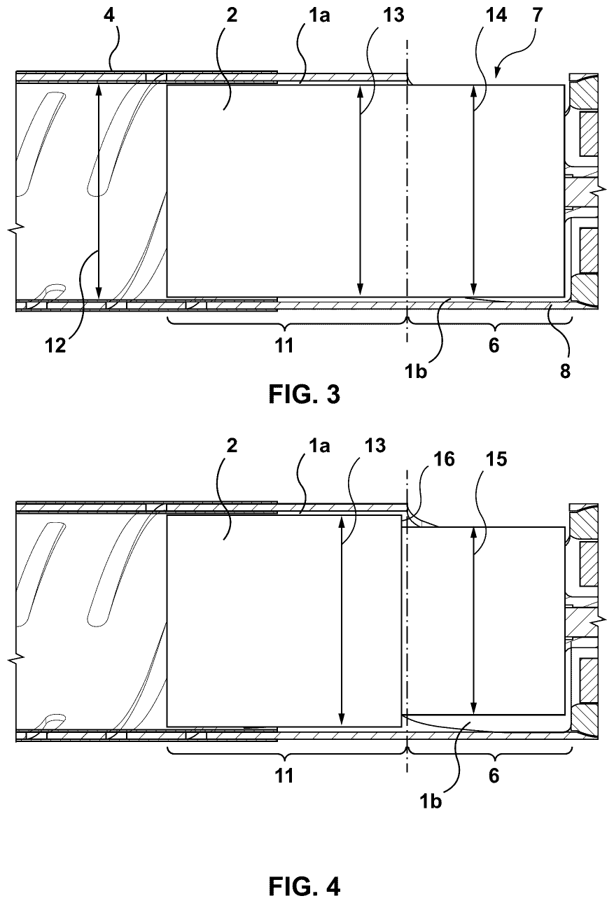

FIG. 3 is a schematic illustration of a cutaway view of a portion of an MCS device, showing an impeller having a consistent impeller diameter, which may be representative of some of the arrangements in current MCS devices. For simplification the impeller 2 is drawn as a cylinder to represent the outer most extent of at least two helically pitched impeller blades and show the diameter 13. The impeller 2 may have more than two impeller blades, preferably balanced radially. The impeller blades may be substantially helical or have a varying pitch or have a plurality of inflections. The tubular cannula 4 may have an inner diameter 12, for example in a range of 3.8 mm to 6 mm (e.g., in a range of 3.8 mm to 5 mm, or in a range of 4.3 mm to 4.5 mm). The impeller has a first diameter 13 in the distal impeller region 11 and a second diameter 14 in the outflow region 6. In FIG. 3 the first diameter 13 and the second diameter 14 are equal resulting in the first gap 1a and the second gap 1b being equal.

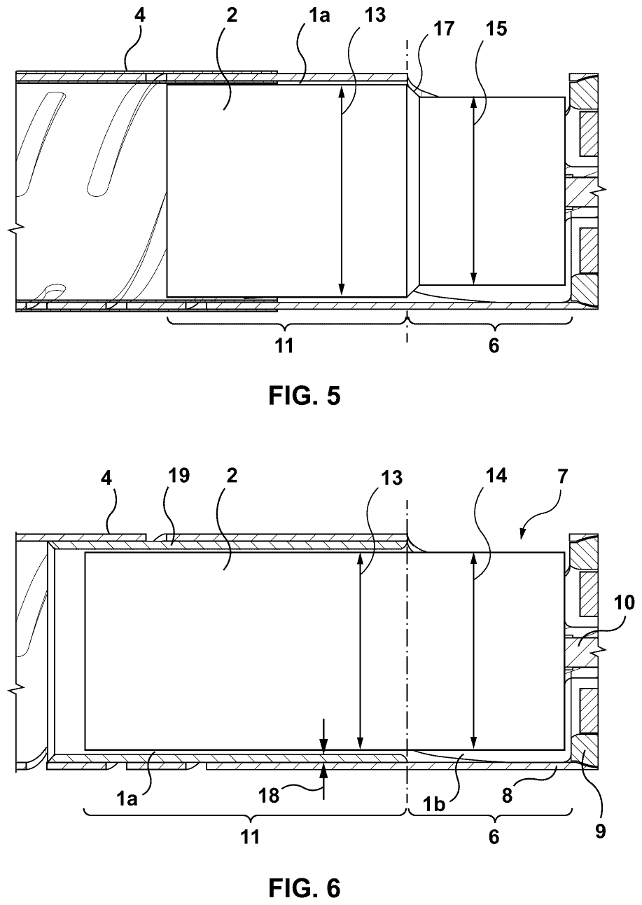

In contrast to FIG. 3, FIG. 4 shows an impeller 2, also drawn as a cylinder for simplification, having a first diameter 13 in the distal impeller region 11, and a second diameter 15 in the outflow region 6, wherein the second diameter 15 is less than the first diameter 13. The resulting first gap 1a is minimalized for optimum efficiency, for example in a range of 0.040 mm to 0.200 mm (e.g., in a range of 0.070 to 0.1 mm, about 0.075 mm), and the second gap 1b is larger than the first gap 1a, for example the second gap 1b may be in a range of 1 to 50 times as large as the first gap 1a (e.g., the second gap 1b may be in a range of 0.040 mm to 2 mm, about 0.20 mm). The difference between the first diameter 13 and reduced diameter 15 may be about 0.25 mm. The transition 16 between the first diameter 13 and second diameter 15 may be a step as shown in FIG. 4. Alternatively, the transition may be a gradual slope 17 as shown in FIG. 5. For example, the slopped transition 17 may have a rise vs. run or change in diameter vs. length of slope in a range of 0.011 to 2 (e.g., 1). Furthermore, the transition may be curved or have multiple inflections (e.g., an S-shaped curve).

An alternative embodiment is shown in FIG. 6, which is a schematic illustration of a portion of an MCS device showing a cutaway view of an impeller 2 having a consistent diameter, in other words the first diameter 13 in the distal impeller region 11 is equal to the second diameter 14 in the outflow region 6. The tubular cannula 4 has a consistent outer diameter in the distal impeller region 11 and the outflow region 6. A sleeve 19 is inserted in the tubular cannula at least in the distal impeller region 11 but not in the outflow region 6. The sleeve 19 is intended to remain stationary in use with respect to the inlet tube. The sleeve has an outer diameter that fits snugly in the inner diameter of the tubular cannula, optionally adhered, form fit, or press fit together, and an inner diameter that is slightly larger than the first impeller diameter 13 by a distance equal to the gap 1a. Since the sleeve 19 is not in the outflow region 6, the gap 1b is larger than gap 1a by the thickness 18 of the sleeve 19, which may be in a range of 0.04 to 2 mm, for example. Optionally, the sleeve 19 has a distal end and a proximal end that are tapered or chamfered as shown to improve fluid flow. Optionally, the sleeve 19 may be made of a high-density polymer such as PEEK to resist wear should the impeller contact the sleeve during use. Optionally, the inner surface of the sleeve may have a lubricious coating to further reduce hydraulic sheer stress in the blood.

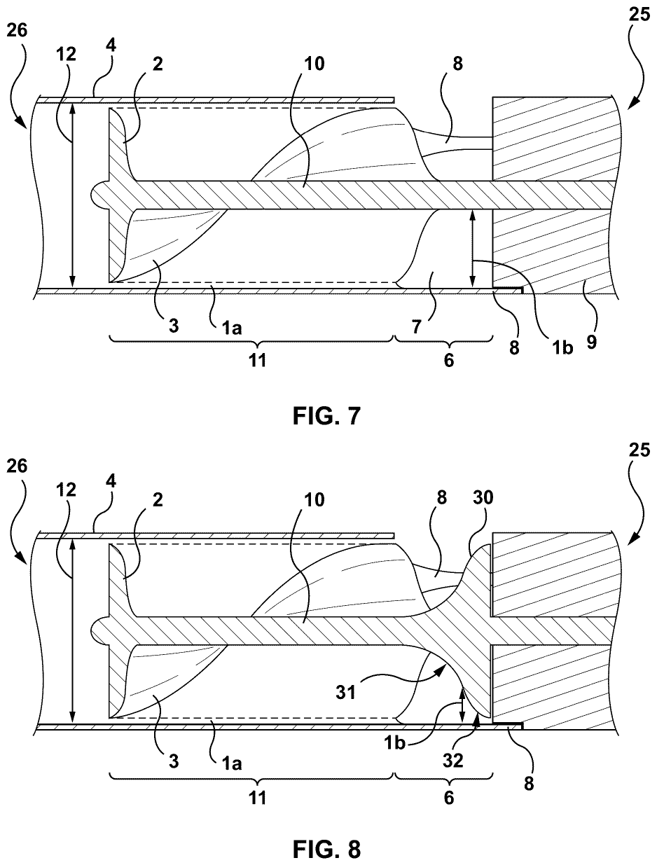

Another embodiment is shown in FIG. 7, a schematic illustration of a portion of an MCS device showing a cutaway view of an impeller 2 having impeller blades 3 in the distal impeller region 11 and no impeller blades in the outflow region 6. The impeller shaft 10 may be rod-shaped in the distal impeller region 11 from which the impeller blades 3 extend radially and the rod-shaped shaft 10 may pass through the outflow region to connect to the motor driveshaft or a rotor. The first radial gap 1a between impeller blades 3 and the inner surface of the tubular cannula 4 may be in a range of 0.04 mm to 0.5 mm (e.g., 0.1 mm). the second radial gap 1b spans the distance between the shaft 10 and the inner surface of the struts 8 and may be in a range of 1.75 mm to 3 mm.

Alternatively, as shown in FIG. 8, the shaft 10 may be rod-shaped in the distal impeller region 11 then have a conical form 30 in the outflow region. Optionally the conical form 30 may have a concave taper 31 that may facilitate directing the blood flow out through the windows 7 in the outflow region 6. Optionally, a proximal portion of the conical part of the shaft 30 has a convex curve 32. The first radial gap 1a between impeller blades 3 and the inner surface of the tubular cannula 4 may be in a range of 0.04 mm to 0.5 mm (e.g., 0.1 mm). The second radial gap 1b is the distance between the conical shaft 30 and the inner surface of the struts 8, wherein the gap 1b at the closest point to the strut 8 may be in a range of 0.04 mm to 2 mm. Optionally, the conical form 30 may have an electropolished surface.

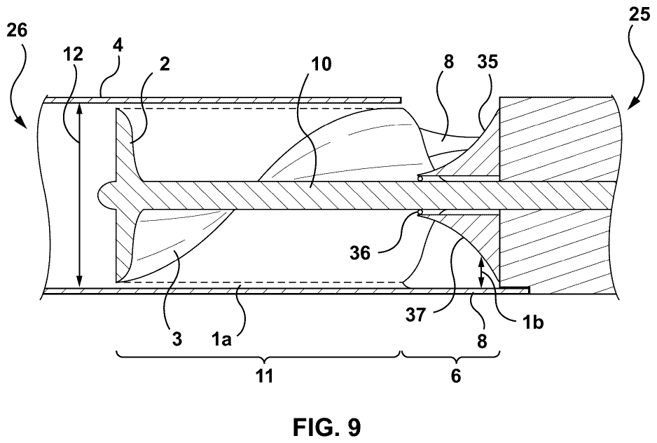

Another embodiment is shown in FIG. 9, a schematic illustration of a portion of an MCS device showing a cutaway view of an impeller 2 having impeller blades 3 in the distal impeller region 11 and no impeller blades in the outflow region 6. Similar to the device shown in FIG. 8, there is a conical form 35 in the outflow region. However, the conical form 35 is not rigidly connected to the impeller shaft 10. Instead, the conical form 35 is stationary with respect to the tubular canula 4 or motor housing, for example the conical form 35 may be rigidly connected to the motor housing or the tubular cannula. The impeller shaft 10 passes through the conical form 35. Optionally, a bearing or seal 36 may be positioned between the impeller shaft 10 and the conical form 35. The conical form may have a concave surface 37 to facilitate directing blood flow out through the windows 7. Optionally, a proximal portion of the conical form 35 may have a convex surface (not shown). The first radial gap 1a between impeller blades 3 and the inner surface of the tubular cannula 4 may be in a range of 0.04 mm to 0.5 mm (e.g., 0.1 mm). In the outflow region a second radial gap between the conical form 35 and inner surface of the struts 8 may be as little as 0, in other words the conical form 35 may connect to the struts 8 at the proximal end and the distance between the conical form 35 and struts 8 may increase moving distally.

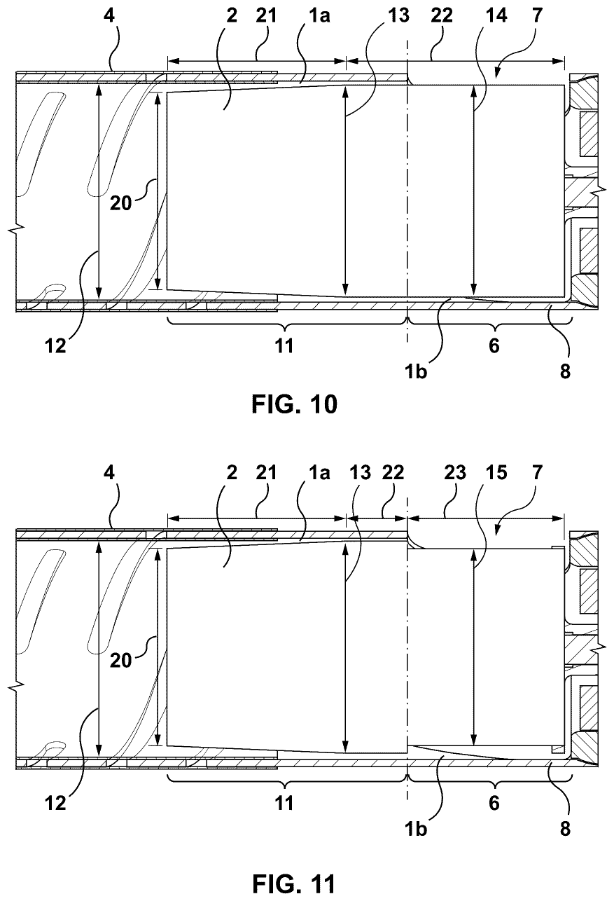

Another embodiment is shown in FIG. 10, a schematic illustration of a cutaway view of a portion of an MCS device showing an impeller region. For simplification, the impeller 2 is shown as an outline of the extent of the impeller's outer diameter. Such outline may refer to a geometric reference volume, which may be cylindrical, that is swept out by the rotating impeller 2. The outline may thus be a maximum diameter of impeller blades 3. The impeller 2 may have two or more of the impeller blades 3, preferably balanced radially. In some embodiments, the impeller 2 may be mounted on a drive shaft 10. As shown in FIG. 10, the impeller 2 is positioned within a tubular cannula 4 having an outflow region 6 and distal impeller region 11, as shown in FIG. 2A. In contrast to other embodiments described herein, the first impeller diameter 13 in at least a portion of the distal region 11 is equal to the second impeller diameter 14 in the outflow region 6. In some embodiments, the distal impeller region 11 may have a distally tapered section 21 that tapers distally to a distal end of the region 11. In other words, the distal impeller region 11 may include a distally tapered section 21 positioned distal to a constant diameter section 22. The constant diameter section 22 can extend proximally from the distally tapered section 21 at least partially within the impeller region 11 and can extend into the outflow region 6.

A first radial gap between the impeller 2 and surrounding tubular structure at a first region may be greater than a second radial gap between the impeller 2 and surrounding tubular structure at a second region, where the second region is located proximal of the first region. A first diameter of the impeller 2 at a first region may be less than a second diameter of the impeller 2 at a second region, where the second region is located proximal of the first region. The distal diameter 20 may be less than the first diameter 13. As used herein, the “diameter,” “width,” “gap,” etc. related to the impeller and a surrounding structure may be measured with respect to the maximum radially outer width of the impeller in that region, which may be an outline of a geometric reference volume swept out by the rotating impeller 2, as described above.

In some embodiments, the diameter of the impeller in the distally tapered section 21 may decrease from a maximum at the first diameter 13 to a relatively smaller distal diameter 20 over the length of the distally tapered section 21. In some embodiments, the tapering of the distally tapered section 21 can prevent contact between the distal aspects of the impeller 2 and the tubular inflow cannula 4. As shown in FIG. 10, in some embodiments, the impeller 2 may be mounted to the drive shaft 10 at its proximal end and not held by a bearing at its distal end. Furthermore, the tubular inflow cannula 4 can be cantilevered from the motor housing. In this configuration there is a possibility of the tubular inflow cannula 4 deflecting slightly, which may be more pronounced in the distal impeller region 11 compared to the outflow region 6. Also, there could be very small imperfections in mounting the impeller 2 with its axis of rotation exactly parallel and aligned with the axis of rotation of the drive shaft 10. These factors may contribute to a risk of contact between the impeller 2 and cannula 4, particularly at the distal end of the impeller 2. The dimensions of the distally tapered section 21 may be chosen to reduce or eliminate the risk of contact while having negligible effects on flow efficiency. These dimensions may depend on the length of the impeller 2 and the inner diameter of the cannula 4.

In one implantation of an MCS device, the impeller 2 has a length between a proximal edge to a distal edge in a range of 7.5 mm to 8.5 mm (e.g., 7.97 mm) and the inlet tube has an inner diameter in a range of 4.39 mm to 4.45 mm (e.g., about 4.42 mm), at least in the region where the impeller 2 is positioned. The difference between the first diameter 13 and the distal diameter 20 may be in a range of 0.040 mm to 0.100 mm (e.g., 0.050 mm to 0.090 mm, 0.060 mm to 0.080 mm, or about 0.070 mm). The first diameter 13 may be about 4.270 mm, the distal diameter 20 may be about 4.200 mm, and the length of the tapered section 21 may be about 3 mm. Thus, the slope of the tapered section 21 may be (0.07 mm/2)/3 mm=0.0117. The resulting radial gap between the impeller 2 and the canula 4 may be constant in the constant diameter section 22, for example in a range of 0.060 to 0.090 mm (e.g., about 0.075 mm), and increase to a gap in a range of 0.095 to 0.125 mm (e.g., about 0.110 mm) at the distal edge of the impeller 2, where the impeller 2 has the distal diameter 20.

Optionally, the distally tapered section 21 may have a taper at a linear slope. Alternatively, the decrease in diameter may be non-linear, for example, a concave, convex or complex curve.

Optionally, as shown in FIG. 11, the impeller 2 may have the distally tapered section 21, and additionally have a proximal narrowed diameter section 23 aligned at least partially in the outflow region 6. This embodiment may have the benefits of decreased risk of contact between the impeller 2 and the cannula 4. This embodiment also may have improved hemolysis in the outflow region 6. The narrowed diameter 15 of the narrowed diameter section 23 of the impeller 2 may be in a range of 0.15 mm to 0.35 mm (e.g., about 0.25 mm) narrower than the first diameter 13. The radial gap between the impeller 2 and cannula 4, or at least the outflow struts 8 at the narrowed diameter section 23 (for example, at the location of the narrowed diameter 15), may be about 0.20 mm, or about 0.125 mm greater than the minimum radial gap in the distal impeller region 11 (for example, at the location of the maximum diameter 13).

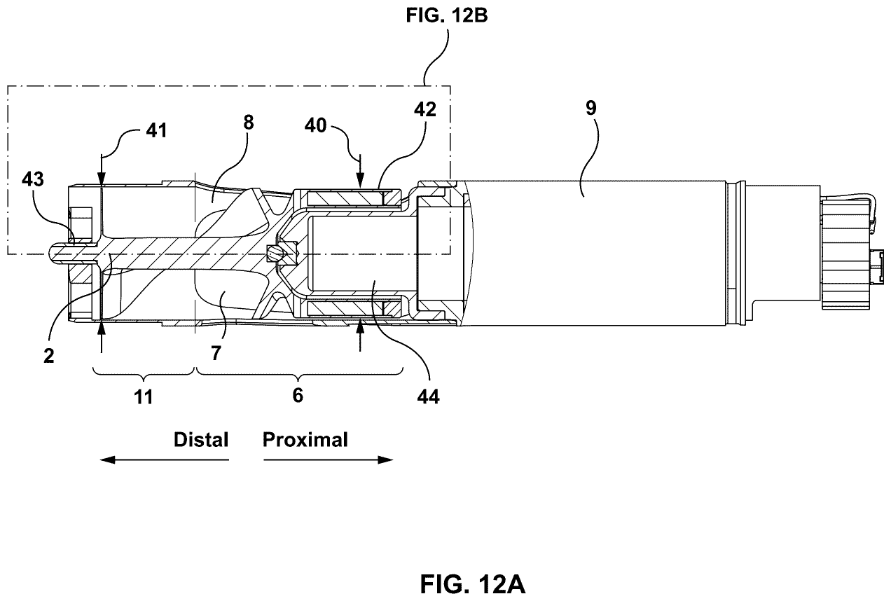

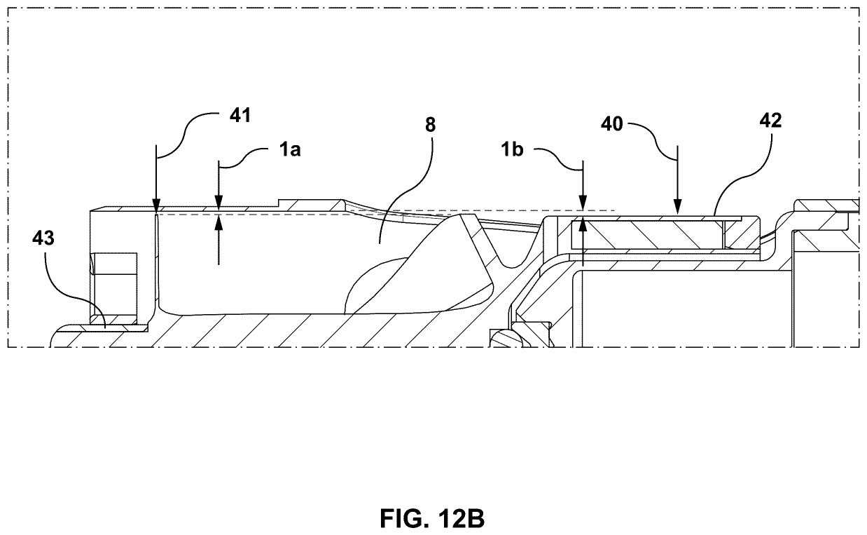

In some embodiments, an MCS device may have an impeller having a different configuration than the one shown in FIG. 2A and have a variable radial gap between the impeller and surrounding cannula or impeller housing. For example, FIG. 12A shows a partial cutaway view of a portion of an MCS device including an impeller 2. A distal end of the impeller 2 may be held in an axial center of a tubular inflow cannula or impeller housing 4 by a bearing 43. The bearing 43 may have spokes that connect it to the cannula or impeller housing 4 maintaining its position with respect to the cannula or impeller housing 4. This may reduce or eliminate the risk of the distal end of the impeller 2 from contacting the cannula or impeller housing 4. In some embodiments, this configuration may benefit from a reduced impeller diameter in the outflow region 6 to reduce hemolysis. Thus, the MCS device may have a first radial gap in the distal impeller region 11 and a second radial gap at least partially in the outflow region 6, wherein the second radial gap is larger than the first radial gap, for example, as described in other embodiments herein. The first radial gap may be about 0.075 mm and the second radial gap may be about 0.200 mm. In some embodiments, the second radial gap may be in a range of two to three times first radial gap.

The impeller 2 may be configured to be coupled to a motor 9 with a contactless magnetic coupling, as shown in FIG. 12. To achieve a magnetic coupling the impeller 2 may by joined to a driven magnetic rotor 42 that surrounds a second magnetic rotor 44 mounted to a drive shaft of the motor 9. Embodiments of examples of such a configuration are disclosed in International PCT Publication Numbers WO 2019/219874, WO 2019/219883, WO 2020/011795, WO 2020/011797, WO 2020/030700, WO 2020/064911, and pending U.S. Provisional Application No. 63/116,616, which are incorporated by reference herein in their entirety. The driven magnetic rotor 42 may be positioned in the outflow region 6 along with a portion of the impeller blades 3. In some embodiments, the cannula or impeller housing 4 surrounding the impeller 2 may have a constant inner diameter. The impeller 2 may have a first diameter 41, and the driven magnetic rotor 42 may have a second diameter 40 that is smaller than the first diameter 41. This may result in a first radial gap 1a around the impeller 2 (e.g., in a range of 0.065 to 0.150 mm, about 0.070 mm to 0.110 mm, or about 0.075 mm) that is smaller than a second radial gap 1b around the driven magnetic rotor 42, which may be in a range of two to three times the first radial gap, as shown in FIG. 12B, a closeup of a portion of FIG. 12A. As shown in FIG. 12A, the first diameter 41 may be positioned within the distal impeller region (11), and the second diameter 40 may be positioned within the outflow region 6. For example, impeller blades may have a maximum outer diameter that changes from a first diameter 41 in the distal impeller region 11 to a second diameter 40 at least partially in the outflow region 6, and/or the impeller blades may have a maximum outer diameter equal to the first diameter 41 in the distal impeller region 11 and the impeller 2 may include a magnetic coupling 42 that has a maximum outer diameter equal to the second diameter 40 positioned at least partially in the outflow region 6.

While the above description provides examples of one or more processes or apparatuses, it will be appreciated that other processes or apparatuses may be within the scope of the accompanying claims.

Specific embodiments described herein are not intended to limit any claim, and any claim may cover processes or apparatuses that differ from those described below, unless specifically indicated otherwise. The claims are not limited to apparatuses or processes having all of the features of any one apparatus or process described below or to features common to multiple or all of the apparatuses described below, unless specifically indicated otherwise. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A mechanical circulatory support device comprising:
a tubular cannula comprising an inflow region, an outflow region, and a distal impeller region, and
an impeller having a proximal end and a distal end, the impeller being positioned within the tubular cannula at least partly in the outflow region and at least partly in the distal impeller region,
wherein a radial gap between the impeller and an inner surface of the tubular cannula varies in size between the proximal end and the distal end, wherein the size of the radial gap at a particular location between the proximal end and the distal end comprises a radial distance between a maximum impeller width and the inner surface of the tubular cannula at the particular location,
wherein the impeller comprises a constant diameter section having a first diameter, and
wherein the impeller comprises a proximal narrowed diameter section proximal to the constant diameter section, the proximal narrowed diameter section having a narrowed diameter less than the first diameter.

2. The device of claim 1, wherein the constant diameter section extends at least partially within the distal impeller region and at least partially into the outflow region.

3. The device of claim 1, wherein the constant diameter section is positioned fully in the distal impeller region.

4. The device of claim 1, wherein the radial gap is between 0.040 mm and 0.200 mm at a location of the first diameter.

5. The device of claim 1, wherein the radial gap is between 0.070 mm and 0.100 mm at the location of the first diameter.

6. The device of claim 1, wherein a transition between the proximal narrowed diameter section and the constant diameter section comprises a taper.

7. The device of claim 6, wherein the taper comprises a linear slope.

8. The device of claim 7, wherein a slope of the linear slope is in a range of 0.011 to 2.

9. The device of claim 1, wherein the narrowed diameter is between 0.15 mm and 0.35 mm less than the first diameter.

10. The device of claim 1, wherein a transition between the proximal narrowed diameter section and the constant diameter section comprises a step transition.

11. The device of claim 1, wherein a transition between the proximal narrowed diameter section and the constant diameter section comprises a gradual slope.

12. The device of claim 11, wherein the gradual slope has a ratio of change in diameter to length of slope of between 0.011 and 2.

13. The device of claim 11, wherein the gradual slope is curved.

14. The device of claim 11, wherein the gradual slope comprises an inflection.

15. The device of claim 14, wherein the gradual slope comprises multiple inflections.

16. The device of claim 1, wherein the proximal narrowed diameter section is positioned entirely in the outflow region.

17. The device of claim 1, wherein the proximal narrowed diameter section has a constant diameter equal to the narrowed diameter.
